(19) United States
(12) Patent Application Publication
(10) Pub. No.: US 2024/0261455 A1
(43) Pub. Date: Aug. 8, 2024

(54) TWO-STAGE UV LIGHTING SYSTEM WITH SAFETY LOCKOUT

(71) Applicant: Evergreen UV, LLC, Memphis, TN (US)

(72) Inventors: Donovan Allen, Oakland, TN (US); Travis Nichols, Memphis, TN (US); David Robert Skelton, Bartlett, TN (US)

(73) Assignee: Evergreen UV, LLC, Memphis, TN (US)

(21) Appl. No.: 18/432,382

(22) Filed: Feb. 5, 2024

Related U.S. Application Data
(60) Provisional application No. 63/443,369, filed on Feb. 3, 2023.

(51) Int. Cl. A61L 9/20

(52) U.S. Cl. CPC A61L 9/20; A61L 2209/12

(57) ABSTRACT

A two-stage UV light system for an air handler includes at least one first stage UV light and at least one second stage UV light. The two-stage UV light system is configured to provide power to the at least one first stage UV light and not the at least one second stage UV light when air is flowing through the air handler, and to provide power to the at least one second stage UV light and not the at least one first stage UV light when air is not flowing through the air handler. The two-stage UV light system can further include a safety lockout system that cuts power to the first and second stage UV lights responsive to a door switch being tripped and/or a safety lockout switch being activated.

### CROSS-REFERENCE TO RELATED APPLICATIONS

This patent application claims priority from, and incorporates by reference the entire disclosure of, U.S. Provisional Patent Application No. 63/443,639 filed on Feb. 3, 2023.

### TECHNICAL FIELD

The present disclosure relates generally to a system for treating air and more particularly, but not by way of limitation, to a two-stage UV lighting system with a safety lockout.

### BACKGROUND

This section provides background information to facilitate a better understanding of the various aspects of the disclosure. It should be understood that the statements in this section of this document are to be read in this light, and not as admissions of prior art.

Heating, ventilation, and air conditioning (“HVAC”) systems are used to control temperatures inside buildings (e.g., homes, schools, commercial buildings, hospitals, etc.). HVAC systems generally include some type of heat exchanger that exchanges heat with air that flows through the exchanger. Recently, there has been increased interest in the ability to not only heat or cool air via the HVAC system, but to also treat the air to remove various pathogens. One method of treatment includes exposing the air that passes through the HVAC system to UV light to irradiate pathogens in the air. This UV treatment method has been shown to be effective, but can require significant power in order to supply enough UV energy to the pathogens due to the limited exposure time of the air to the UV light sources. Lowering the power usage of air handling systems that utilize UV light to treat air would result in reduced operating costs.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

A two-stage UV light system for an air handler includes at least one first stage UV light and at least one second stage UV light. The two-stage UV light system is configured to provide power to the at least one first stage UV light and not the at least one second stage UV light when air is flowing through the air handler, and to provide power to the at least one second stage UV light and not the at least one first stage UV light when air is not flowing through the air handler. The two-stage UV light system can further include a safety lockout system that cuts power to the first and second stage UV lights responsive to a door switch being tripped and/or a safety lockout switch being activated.

In some aspects, the two-stage UV light system further incudes a safety lockout system with a lockout switch configured to cut power to the at least one first and second stage UV lights; and a door switch configured to trip when a door of the air handler is opened, the door switch being electrically coupled to the lockout switch. The safety lockout system is configured to cut power to the at least one first and second stage UV lights when either the lockout switch is activated or the door switch is tripped.

In some aspects, the at least one first stage UV light operates at a first power level and the at least one second stage UV light operates at a second power level that is lower than the first power level, the first power level being used for when air moves through the air handler and the second power level being used for when air is not moving through the air handler.

In some aspects, the at least one first and second stage UV lights are a single UV light that is configured to operate at a first power level and a second power level.

A safety lockout system for an HVAC system includes a control panel; a lockout switch associated with the control panel; and a door switch configured to trip when a door is opened, the door switch being electrically coupled to the control panel. The safety lockout system is configured to cut power to a UV light within the HVAC system when either the lockout switch is activated or the door switch is tripped.

In some aspects, the lockout switch is configured to activate when the door switch is triggered.

In some aspects, the lockout switch is configured to remain activated to cut power to the UV light after the door is closed.

A method of treating air within an HVAC system includes flowing air through an air handler of the HVAC system and exposing an interior of the air handler to a first amount of UV light from at least one UV light source; stopping the air flowing through the air handler and exposing the interior of the air handler to a second amount of UV light from the at least one UV light source that is lower than the first power level.

In some aspects, the method includes cutting power to the at least one UV light source responsive to a door switch of the air handler being tripped.

In some aspects, tripping the door switch activates a lockout switch that prevents power from being supplied to the at least one UV light source until the lockout switch has been deactivated.

In some aspects, the method includes cutting power to the at least one UV light source responsive to activation of a lockout switch.

In some aspects, the at least one UV light source comprises a first plurality of UV lights configured to operate at a first power level for supplying the first amount of UV light and a second plurality of UV lights configured to operate at second power level for supplying the second amount of UV light.

In some aspects, the at least one UV light source comprises a plurality off UV lights configured to operate at a first power level for supplying the first amount of UV light and a second power level for supplying the second amount of UV light.

In some aspects, the at least one UV light source comprises a plurality of UV lights configured so that a first number of the plurality of UV lights are operated to supply the first amount of UV light and a second number of the plurality of UV lights are operated to supply the second amount of UV light, the second number being less than the first number.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 is a schematic illustrating a two-stage UV lighting system with a safety lockout, according to aspects of the disclosure.

FIGS. 2A and 2B illustrate front and side views, respectively, of a two-stage UV lighting system, according to aspects of the disclosure.

FIG. 3 is a schematic illustrating a safety lockout system according to aspects of the disclosure.

FIG. 4 is a schematic illustrating a computer system/controller for a two-stage UV lighting system with a safety lockout, according to aspects of the disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described. Reference will now be made to more specific embodiments of the present disclosure and data that provides support for such embodiments. However, it should be noted that the disclosure below is for illustrative purposes only and is not intended to limit the scope of the claimed subject matter in any way.

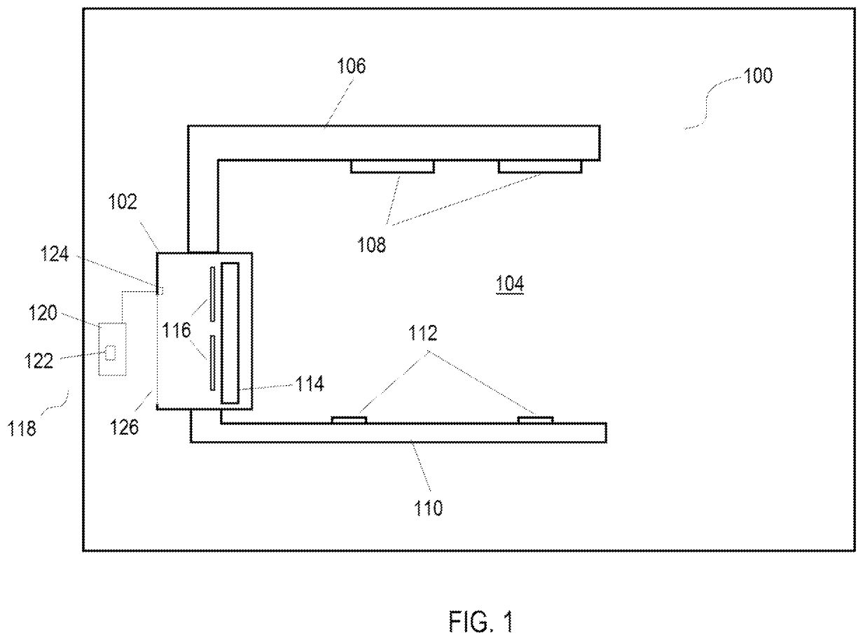

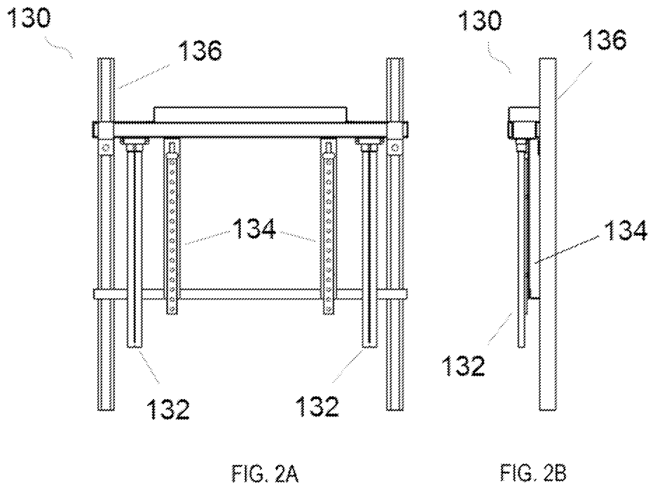

FIG. 1 is a schematic illustrating a two-stage UV lighting system 100 for an HVAC system according to aspects of the disclosure. System 100 is shown positioned within a space 104 (e.g., a building that could be a school, commercial space, industrial space, home, etc.). The HVAC system includes an air handler 102, an air duct 106, air vents 108, an air duct 110, return air vents 112. HVAC system 100 may include other components that are not shown in FIG. 1 (e.g., compressors, controllers, etc.). Air handler 102 is configured to receive air from a space 104 via air duct 110. That air is then passed through a heat exchanger 114 to condition the air (e.g., to cool the air). A plurality of UV lights 116 are positioned within air handler 102 to be proximal to heat exchanger 114 to expose the air that has passed through heat exchanger 114 and heat exchanger 114 itself to UV light to irradiate pathogens in the air and on the surfaces of air handler 102 and heat exchanger 114. UV lights 116 may comprise system 130 of FIGS. 2A and 2B, described in more detail below. UV lights 116 are selected to be effective in irradiating pathogens. In some aspects, UV lights 116 are selected to emit a wavelength in the range of about 100 nm to about 400 nm. The treated air then travels through air duct 106 to air vents 108 where it is expelled into space 104. The air continues to cycle through space 104 as desired to both control the temperature of the air in space 104, and to eliminate pathogens from the air in space 104.

In some aspects, system 100 includes a safety lockout system 118. Safety lockout system 118 includes a control panel 120 with a lockout switch 122, and a door switch 124. Safety lockout system 118 is configured to shut off power to the plurality of UV lights 116 to prevent a person from being exposed to UV light when the person is inside air handler 102. Safety system 118 operates in two different ways. In a first mode, the person who is entering air handler 102 activates lockout switch 122, which cuts power to the plurality of UV lights 116. The person may then open a door or access panel 126 to enter air handler 102. Power will remain cut off from the plurality of UV lights 116 until lockout switch 122 is deactivated. In a second mode, power to the plurality of UV lights 116 is cut even if the person does not activate lockout switch 122. In this mode, when the user opens door 126, door switch 124 is tripped. Door switch 124 is electrically coupled to control panel 120 and is configured as a redundant power cut off switch for the plurality of UV lights 116. Tripping door switch 124 cuts power to the plurality of UV lights 116 until lockout switch 122 is deactivated. In other words, even if door 126 is closed (e.g., with the person still inside), power to the plurality of UV lights 116 will remain cut until lockout switch 122 is deactivated.

FIGS. 2A and 2B illustrate front and side views, respectively, of a two-stage UV lighting frame system 130 according to aspects of the disclosure. System 130 includes a first UV light 132 and a second UV light 134 (illustrated as a pair of UV lights 134, but in other aspects a single UV light or three or more UV lights may be used). UV lights 132, 134 are shown secured to a frame 136. Frame 136 is configured to be secured within an air handler (e.g., air handler 102) and proximal to a heat exchanger (e.g., heat exchanger 114) of the air handler. Depending on the size of the air handler and heat exchanger, multiple systems 130 may be installed as needed (e.g., multiple systems 130 connected in series or parallel). A single system 130 will be discussed below with the understanding that the discussion applies to an HVAC system that includes multiple systems 130.

First UV light 132 is a first-stage UV light that is a “high energy” UV light capable of providing enough UV energy to effectively irradiate pathogens in the air that flows through the heat exchanger. Second UV light 134 is a second-stage UV light that is a “low energy” UV light that provides less energy than first UV light 132. Second UV light 134 may be, for example, an LED UV light that consumes less power (e.g., 40-60% less power) than first UV light 132. In a typical aspect, second UV light 134 is not capable of providing enough energy to irradiate air flowing through the air handler. Instead, second UV light 134 is configured to operate in place of first UV light 132 when air is not moving through the air handler. When air is not moving through the air handler, second UV light 134 provides enough energy to irradiate pathogens in the air within the air handler and pathogens that may have settled on surfaces within the air handler (e.g., on the heat exchanger and the walls of the handler etc.).

Compared to a system that only includes first UV light 132, a system having both first UV light 132 and second UV light 134 reduces the amount of energy consumed by the system. For example, a single stage system must either run all the time—even when the air is not flowing through the air handler—to prevent pathogen build up, or must be turned off when the air is not flowing through the air handler. In the first instance, a considerable amount of power is being wasted when no air is flowing (i.e., considerably less power is needed to irradiate non-moving air). In the second instance, pathogens are allowed to grow and spread within the air handler and on the heat exchanger when the UV lights are powered off, which is not desirable. The two-stage system described herein overcomes both of these limitations. For example, first UV light 132 can be shut off when no air is flowing through the air handler and second UV light 134 is powered on. Second UV light 134 provides enough energy to properly irradiate the inside of the air handler and the heat exchanger when there is no air flow through the air handler. When the air begins to flow through the air handler, second UV light 134 is shut off and first UV light 132 is powered on. Thus, the two-stage UV lighting system both reduces power consumption by the system and provides irradiation of pathogens when no air is flowing through the system.

In some aspects, first UV light 132 may itself be a two-stage light. For example, first UV light 132 may be operable in at least two states—a high-powered state and a low-powered state. In the high-powered state, first UV light 132 supplies sufficient energy to effectively irradiate pathogens in moving air. In the low-powered state, first UV light 132 does not supply sufficient UV energy to irradiate pathogens in moving air but does provide sufficient UV energy to irradiate pathogens in non-moving air. In this aspect, first UV light 132 is operated in the high-powered state when air is flowing through the air handler and is operated in the low-powered state when air is not flowing through the air handler.

In some aspects, the system includes a plurality of first UV lights 132 but no second UV lights 134. To reduce power consumption when no air is flowing through the air handler, the system shuts off some of the plurality of first UV lights 132, but not all of the first UV lights 132. For example, in an array having two rows of eight first UV lights 132, at least one of the eight first UV lights 132 may be turned off. More particular, half of the UV lights on the top row and half of the lights on the bottom row may be turned off (or any other configuration/combination of lights that are turned on and turned off may be used). This configuration reduces power consumption by the system.

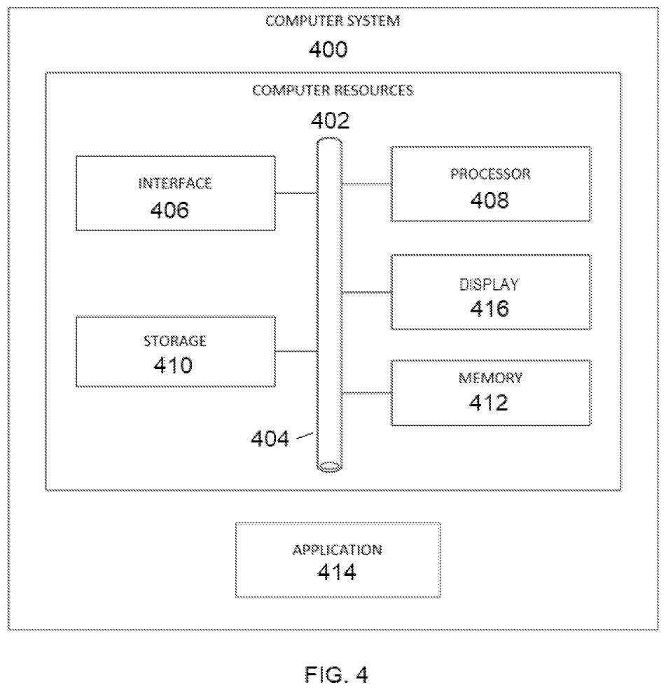

System 130 may be controlled in various ways. In some aspects, system 130 includes a controller that is configured to detect when air is flowing through the air handler. For example, the controller may be tied into the HVAC system to determine when the HVAC system is operating. In other examples, the controller may monitor the fans or blower of the HVAC system to determine when power is being supplied to the fans or blower. The controller is electrically coupled to the UV lights to turn them off and on as desired. FIG. 4, and the related discussion below, describe a computer system/controller that may be used in conjunction with system 100 in more detail.

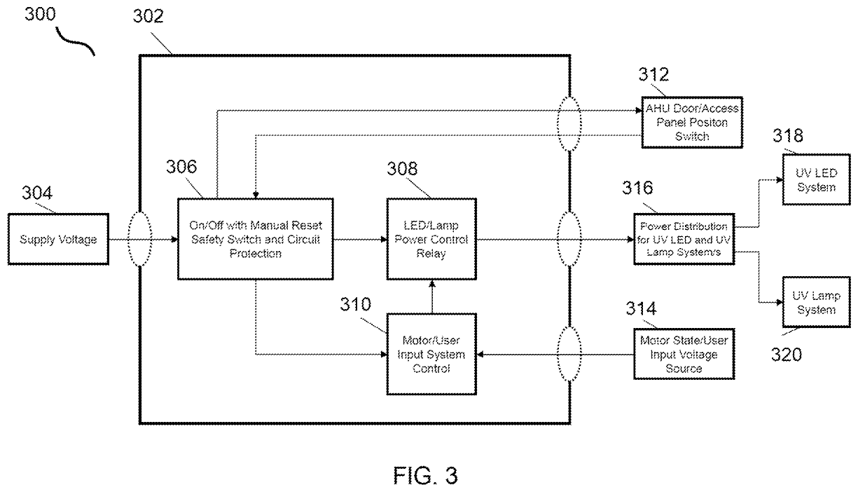

FIG. 3 is a schematic illustrating a safety lockout system 300 according to aspects of the disclosure. System 300 includes a control box 302 that is supplied power from a supply voltage 304. Control box 302 includes a switch 306, an LED power control relay 308, and an input control system 310. Switch 306 is configured to output a voltage from supply voltage 304 to a door panel position switch 312, and to receive a return voltage therefrom. Switch 306 is further configured to output a voltage from supply voltage 304 to LED power control relay 308 and input control system 310. LED power control relay 308 additionally receives voltage from input control system 310 and outputs a voltage to a power distributor 316. Input control system 310 additionally receives a voltage from a motor state/user input voltage source 314 indicating when the motor is operating/or a user specified condition. Power distributor 316 is configured to distribute voltage to UV LED system 318 and/or UV lamp system 320. Safety lockout system 300 may be communicatively coupled with the controller discussed above and described in more detail below relative to FIG. 4.

FIG. 4 is a schematic illustrating a computer system/controller 400 for use with a two-stage UV lighting system with a safety lockout, such as system 100, according to aspects of the disclosure. Computer system 400 includes an application 414 operable to execute on computer resources 402. Application 414 can be, for example, an interface for operating computer system 400. In other embodiments, application 414 can be, for example, an interface for operating and/or accessing all engines and datastores of computer system 400. In particular embodiments, computer system 400 may perform one or more steps of one or more methods described or illustrated herein. In particular embodiments, one or more computer systems may provide functionality described or illustrated herein. In particular embodiments, encoded software running on one or more computer systems may perform one or more steps of one or more methods described or illustrated herein or provide functionality described or illustrated herein.

The components of computer system 400 may comprise any suitable physical form, configuration, number, type and/or layout. As an example, and not by way of limitation, computer system 400 may comprise an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a wearable or body-borne computer, a server, or a combination of two or more of these. Where appropriate, computer system 400 may include one or more computer systems; be unitary or distributed; span multiple locations; span multiple machines; or reside in a cloud, which may include one or more cloud components in one or more networks.

In the depicted embodiment, computer system 400 includes a processor 408, memory 412, storage 410, a display 416, interface 406, and bus 404. Although a particular computer system is depicted having a particular number of particular components in a particular arrangement, this disclosure contemplates any suitable computer system having any suitable number of any suitable components in any suitable arrangement.

Processor 408 may be a microprocessor, controller, or any other suitable computing device, resource, or combination of hardware, software and/or encoded logic operable to execute, either alone or in conjunction with other components, (e.g., memory 412), the application 414. Such functionality may include providing various features discussed herein. In particular embodiments, processor 408 may include hardware for executing instructions, such as those making up the application 414. As an example, and not by way of limitation, to execute instructions, processor 408 may retrieve (or fetch) instructions from an internal register, an internal cache, memory 412, or storage 410; decode and execute them; and then write one or more results to an internal register, an internal cache, memory 412, or storage 410. Display 416 is configured to display information to a user (e.g., route guidance information). In some aspects, display 416 may be a touchscreen display that receives input from a user (e.g., origin and our destination information). In some aspects, display 416 may be integrated into computer system 400 (i.e., when computer system 400 is a mobile, stand-alone unit). In some aspects, display 416 may be integrated into a vehicle (i.e., a part of the infotainment and/or navigation system of an automobile in which computer system 400 is installed).

In particular embodiments, processor 408 may include one or more internal caches for data, instructions, or addresses. This disclosure contemplates processor 408 including any suitable number of any suitable internal caches, where appropriate. As an example, and not by way of limitation, processor 408 may include one or more instruction caches, one or more data caches, and one or more translation lookaside buffers (TLBs). Instructions in the instruction caches may be copies of instructions in memory 412 or storage 410 and the instruction caches may speed up retrieval of those instructions by processor 408. Data in the data caches may be copies of data in memory 412 or storage 410 for instructions executing at processor 408 to operate on; the results of previous instructions executed at processor 408 for access by subsequent instructions executing at processor 408, or for writing to memory 412, or storage 410; or other suitable data. The data caches may speed up read or write operations by processor 408. The TLBs may speed up virtual-address translations for processor 408. In particular embodiments, processor 408 may include one or more internal registers for data, instructions, or addresses. Depending on the embodiment, processor 408 may include any suitable number of any suitable internal registers, where appropriate. Where appropriate, processor 408 may include one or more arithmetic logic units (ALUs); be a multi-core processor; include one or more processors 408; or any other suitable processor.

Memory 412 may be any form of volatile or non-volatile memory including, without limitation, magnetic media, optical media, random access memory (RAM), read-only memory (ROM), flash memory, removable media, or any other suitable local or remote memory component or components. In particular embodiments, memory 412 may include random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM, or any other suitable type of RAM or memory. Memory 412 may include one or more memories 412, where appropriate. Memory 412 may store any suitable data or information utilized by computer system 400, including software embedded in a computer readable medium, and/or encoded logic incorporated in hardware or otherwise stored (e.g., firmware). In particular embodiments, memory 412 may include main memory for storing instructions for processor 408 to execute or data for processor 408 to operate on. In particular embodiments, one or more memory management units (MMUs) may reside between processor 408 and memory 412 and facilitate accesses to memory 412 requested by processor 408.

As an example, and not by way of limitation, computer system 400 may load instructions from storage 410 or another source (such as, for example, another computer system) to memory 412. Processor 408 may then load the instructions from memory 412 to an internal register or internal cache. To execute the instructions, processor 408 may retrieve the instructions from the internal register or internal cache and decode them. During or after execution of the instructions, processor 408 may write one or more results (which may be intermediate or final results) to the internal register or internal cache. Processor 408 may then write one or more of those results to memory 412. In particular embodiments, processor 408 may execute only instructions in one or more internal registers or internal caches or in memory 412 (as opposed to storage 410 or elsewhere) and may operate only on data in one or more internal registers or internal caches or in memory 412 (as opposed to storage 410 or elsewhere).

In particular embodiments, storage 410 may include mass storage for data or instructions. As an example, and not by way of limitation, storage 410 may include a hard disk drive (HDD), a floppy disk drive, flash memory, an optical disc, a magneto-optical disc, magnetic tape, or a Universal Serial Bus (USB) drive or a combination of two or more of these. Storage 410 may include removable or non-removable (or fixed) media, where appropriate. Storage 410 may be internal or external to computer system 400, where appropriate. In particular embodiments, storage 410 may be non-volatile, solid-state memory. In particular embodiments, storage 410 may include read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. Storage 410 may take any suitable physical form and may comprise any suitable number or type of storage. Storage 410 may include one or more storage control units facilitating communication between processor 408 and storage 410, where appropriate.

In particular embodiments, interface 406 may include hardware, encoded software, or both providing one or more interfaces for communication (such as, for example, packet-based communication) among any networks, any network devices, and/or any other computer systems. As an example, and not by way of limitation, communication interface 406 may include a network interface controller (NIC) or network adapter for communicating with an Ethernet or other wire-based network and/or a wireless NIC (WNIC) or wireless adapter for communicating with a wireless network.

Depending on the embodiment, interface 406 may be any type of interface suitable for any type of network for which computer system 400 is used. As an example, and not by way of limitation, computer system 400 can include (or communicate with) an ad-hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 400 can include (or communicate with) a wireless PAN (WPAN) (such as, for example, a BLUETOOTH WPAN), a WI-FI network, a WI-MAX network, an LTE network, an LTE-A network, a cellular telephone network (such as, for example, a Global System for Mobile Communications (GSM) network), or any other suitable wireless network or a combination of two or more of these. computer system 400 may include any suitable interface 406 for any one or more of these networks, where appropriate.

In some embodiments, interface 406 may include one or more interfaces for one or more I/O devices. One or more of these I/O devices may enable communication between a person and computer system 400. As an example, and not by way of limitation, an I/O device may include a keyboard, keypad, microphone, monitor, mouse, printer, scanner, speaker, still camera, stylus, tablet, touchscreen, trackball, video camera, another suitable I/O device or a combination of two or more of these. An I/O device may include one or more sensors. Particular embodiments may include any suitable type and/or number of I/O devices and any suitable type and/or number of interfaces 406 for them. Where appropriate, interface 406 may include one or more drivers enabling processor 408 to drive one or more of these I/O devices. Interface 406 may include one or more interfaces 406, where appropriate.

Bus 404 may include any combination of hardware, software embedded in a computer readable medium, and/or encoded logic incorporated in hardware or otherwise stored (e.g., firmware) to couple components of computer system 400 to each other. As an example and not by way of limitation, bus 404 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCI-X) bus, a serial advanced technology attachment (SATA) bus, a Video Electronics Standards Association local (VLB) bus, or any other suitable bus or a combination of two or more of these. Bus 404 may include any number, type, and/or configuration of buses 404, where appropriate. In particular embodiments, one or more buses 404 (which may each include an address bus and a data bus) may couple processor 408 to memory 412. Bus 404 may include one or more memory buses.

Herein, reference to a computer-readable storage medium encompasses one or more tangible computer-readable storage media possessing structures. As an example and not by way of limitation, a computer-readable storage medium may include a semiconductor-based or other integrated circuit (IC) (such, as for example, a field-programmable gate array (FPGA) or an application-specific IC (ASIC)), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, a flash memory card, a flash memory drive, or any other suitable tangible computer-readable storage medium or a combination of two or more of these, where appropriate.

Particular embodiments may include one or more computer-readable storage media implementing any suitable storage. In particular embodiments, a computer-readable storage medium implements one or more portions of processor 408 (such as, for example, one or more internal registers or caches), one or more portions of memory 412, one or more portions of storage 410, or a combination of these, where appropriate. In particular embodiments, a computer-readable storage medium implements RAM or ROM. In particular embodiments, a computer-readable storage medium implements volatile or persistent memory. In particular embodiments, one or more computer-readable storage media embody encoded software. #

Herein, reference to encoded software may encompass one or more applications, bytecode, one or more computer programs, one or more executables, one or more instructions, logic, machine code, one or more scripts, or source code, and vice versa, where appropriate, that have been stored or encoded in a computer-readable storage medium. In particular embodiments, encoded software includes one or more application programming interfaces (APIs) stored or encoded in a computer-readable storage medium. Particular embodiments may use any suitable encoded software written or otherwise expressed in any suitable programming language or combination of programming languages stored or encoded in any suitable type or number of computer-readable storage media. In particular embodiments, encoded software may be expressed as source code or object code. In particular embodiments, encoded software is expressed in a higher-level programming language, such as, for example, C, Perl, or a suitable extension thereof. In particular embodiments, encoded software is expressed in a lower-level programming language, such as assembly language (or machine code). In particular embodiments, encoded software is expressed in JAVA. In particular embodiments, encoded software is expressed in Hyper Text Markup Language (HTML), Extensible Markup Language (XML), or other suitable markup language.#

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.#

Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth herein.#

What is claimed is:

1. A two-stage UV light system configured to be disposed within an air handler, the two-stage UV light system comprising:
at least one first stage UV light and at least one second stage UV light, and
wherein the two-stage UV light system is configured to provide power to the at least one first stage UV light and not the at least one second stage UV light when air is flowing through the air handler, and to provide power to the at least one second stage UV light and not the at least one first stage UV light when air is not flowing through the air handler.

2. The system of claim 1, further comprising a safety lockout system comprising:
a lockout switch configured to cut power to the at least one first and second stage UV lights; and
a door switch configured to trip when a door of the air handler is opened, the door switch being electrically coupled to the lockout switch, and
wherein the safety lockout system is configured to cut power to the at least one first and second stage UV lights when either the lockout switch is activated or the door switch is tripped.

3. The system of claim 1, wherein the at least one first stage UV light operates at a first power level and the at least one second stage UV light operates at a second power level that is lower than the first power level, the first power level being used for when air moves through the air handler and the second power level being used for when air is not moving through the air handler.

4. The system of claim 1, wherein the at least one first and second stage UV lights are a single UV light that is configured to operate at a first power level and a second power level.

5. A safety lockout system for an HVAC system, the safety lockout system comprising:
a control panel;
a lockout switch associated with the control panel; and
a door switch configured to trip when a door is opened, the door switch being electrically coupled to the control panel, and
wherein the safety lockout system is configured to cut power to a UV light within the HVAC system when either the lockout switch is activated or the door switch is tripped.

6. The system of claim 5, wherein the lockout switch is configured to activate when the door switch is triggered.

7. The system of claim 6, wherein the lockout switch is configured to remain activated to cut power to the UV light after the door is closed.

8. A method of treating air within an HVAC system, the method comprising:
flowing air through an air handler of the HVAC system and exposing an interior of the air handler to a first amount of UV light from at least one UV light source;
stopping the air flowing through the air handler and exposing the interior of the air handler to a second amount of UV light from the at least one UV light source that is lower than the first power level.

9. The method of claim 8, further comprising cutting power to the at least one UV light source responsive to a door switch of the air handler being tripped.

10. The method of claim 9, wherein tripping the door switch activates a lockout switch that prevents power from being supplied to the at least one UV light source until the lockout switch has been deactivated.

11. The method of claim 8, further comprising cutting power to the at least one UV light source responsive to activation of a lockout switch.

12. The method of claim 8, wherein the at least one UV light source comprises a first plurality of UV lights configured to operate at a first power level for supplying the first amount of UV light and a second plurality of UV lights configured to operate at second power level for supplying the second amount of UV light.

13. The method of claim 8, wherein the at least one UV light source comprises a plurality off UV lights configured to operate at a first power level for supplying the first amount of UV light and a second power level for supplying the second amount of UV light.

14. The method of claim 8, wherein the at least one UV light source comprises a plurality of UV lights configured so that a first number of the plurality of UV lights are operated to supply the first amount of UV light and a second number of the plurality of UV lights are operated to supply the second amount of UV light, the second number being less than the first number.
